(19) United States
(12) Patent Application Publication
(10) Pub. No.: US 2005/0032740 A1
(43) Pub. Date: Feb. 10, 2005

(54) Vitamin compositions for the treatment and prevention of vascular disease and dementia

(76) Inventor: Balaji Venkataraman, Alpharetta, GA (US)

Correspondence Address: JOHN S. PRATT, ESQ;KILPATRICK STOCKTON, LLP, 1100 PEACHTREE STREET, ATLANTA, GA, 30309, US

(21) Appl. No.: 10/635,428

(22) Filed: Aug. 6, 2003

(51) Int. Cl. A61K 31/714; A61K 31/525; A61K 31/355; A61K 33/14

(52) U.S. Cl. 514/52; 514/251; 514/458; 424/681

(57) ABSTRACT

Provided are vitamin compositions and methods for the treatment of dementia and vascular diseases, including cardiovascular diseases. The vitamin compositions comprise vitamin B12, vitamin B6, folic acid, magnesium and vitamin E.

### FIELD OF THE INVENTION

The present invention relates generally to the fields of nutrition and disease treatment. Specifically, the invention relates to vitamin compositions comprising vitamin B12, vitamin B6, folic acid, magnesium, and vitamin E.

### BACKGROUND OF THE INVENTION

Recent evidence has shown a very high prevalence of sub-optimal vitamin levels in diets (Flood et al., J. Natl. Cancer Inst. 2000; 92:1706). A recent article in the Journal of the American Medical Association concluded, “it appears to be prudent for all adults to take vitamin supplementation” (Fletcher et al., JAMA 2002, 287(23):3127). While poor dietary intake of vitamins remains the primary reason for this prevalence, many vitamins can be depleted due to other factors such as alcohol consumption, smoking, genetics, environmental factors, and consumption of certain prescription medications. Certain vitamin deficiencies have also been linked with chronic diseases including vascular diseases, osteoporosis and cancer. Vascular diseases include but are not limited to coronary artery disease, cerebrovascular disease and peripheral vascular disease and may be caused by many different factors. Vascular disease has also been associated with dementia and other diseases of the nervous system including Alzheimer's disease.

Coronary artery disease refers to the narrowing of the coronary arteries that supply blood to the heart. Coronary artery disease is a progressive disease that increases the risk for myocardial infarction (MI) and sudden death. Known risk factors that increase the risk of coronary artery disease include but are not limited to: 1) cigarette smoking 2) dyslipidemia, 3) hypertension, 4) hyperhomocystineamia, 5) genetics and familial history, 6) increasing age, 7) lack of exercise, 8) gender, 9) being overweight or obese and 10) inflammation. These risk factors are also related to diseases in other aspects of the vascular system, such as the cerebrovascular system, the aorta, renal arteries, the carotid arteries and peripheral arteries, and are associated with atherosclerosis, stroke, peripheral vascular disease and claudication.

Accordingly, there is a need for non-invasive vitamin-based treatments and preventatives for vascular diseases.

### SUMMARY OF THE INVENTION

The present invention addresses the problems described above by providing novel vitamin compositions and methods for the treatment and prevention of vascular diseases and dementia.

The vitamin compositions of the present invention comprise the combination of vitamin B12, vitamin B6, folic acid, magnesium and vitamin E. The vitamin compositions may contain any combination of the following amounts of the specific ingredients: magnesium in an amount between about 50 mg and 350 mg, preferably between about 75 mg and 150 mg, and more preferably about 100 mg; vitamin E in an amount less than 400 IU, preferably between about 50 IU and 350 IU, and more preferably about 100 IU; folic acid in an amount between about 0.8 mg and 5 mg, preferably between about 1 mg and 3 mg, and more preferably about 2 mg or about 2.05 mg; vitamin B12 in an amount between about 500 mcg and 2000 mcg, preferably between about 300 mcg and 1200 mcg, and more preferably about 500 mcg; and vitamin B6 in an amount between about 10 mg and 100 mg, and preferably about 25 mg. In some embodiments, the vitamin compositions further comprise niacin in any amount or in an amount less than 250 mg, preferably between about 20 mg and 100 mg, and more preferably about 35 mg.

In one embodiment of the present invention, the vitamin composition contains vitamin B12, vitamin B6, folic acid, magnesium, and vitamin E in any amount. It is to be understood that vitamin B12 may be present in the form of cyanocobalamin, hydroxocobalamin or combinations thereof. In one preferred embodiment, the vitamin composition contains folic acid in an amount of about 2.05 mg, vitamin B12 (hydroxocobalamin) in an amount of about 500 mcg, vitamin B6 (pyridoxine) in an amount of about 25 mg, vitamin E (alpha-tocopherol) in an amount of about 100 IU, and magnesium (as oxide) in an amount of about 100 mg. In another preferred embodiment, the vitamin composition contains folic acid in an amount of about 2.05 mg, vitamin B12 (cyanocobalamin) in an amount of about 500 mcg, vitamin B6 (pyridoxine) in an amount of about 25 mg, vitamin E (alpha-tocopherol) in an amount of about 100 IU, and magnesium (as oxide) in an amount of about 100 mg. In yet another preferred embodiment, the vitamin composition contains folic acid in an amount of about 2.05 mg, vitamin B12 (cyanocobalamin in an amount from 1 to 499 mcg and hydroxocobalamin in an amount from 1 to 499 mcg provided that the total amount of cyanocobalamin and hydroxocobalamin does not exceed 500 mcg), vitamin B6 (pyridoxine) in an amount of about 25 mg, vitamin E (alpha-tocopherol) in an amount of about 100 IU, and magnesium (as oxide) in an amount of about 100 mg. In yet another preferred embodiment, the vitamin composition contains folic acid in an amount of about 2 mg, vitamin B12 (cyanocobalamin in an amount of about 250 mcg and hydroxocobalamin in an amount of about 250 mcg), vitamin B6 (pyridoxine) in an amount of about 25 mg, vitamin E (alpha-tocopherol) in an amount of about 100 IU, and magnesium (as oxide) in an amount of about 100 mg. In further embodiments, the vitamin compositions further comprise about 35 mg of niacin.

The present invention further includes methods of treating or preventing a vascular disease or dementia in an individual by administering to the individual an effective amount of a vitamin composition comprising vitamin B12, vitamin B6, folic acid, magnesium and vitamin E. In one embodiment, the vitamin composition administered to the individual contains vitamin B12, vitamin B6, folic acid, magnesium and vitamin E in any amount. In a preferred embodiment, the B12 vitamin is a cyanocobalamin. In another preferred embodiment, the B12 vitamin is a hydroxocobalamin. In still another preferred embodiment, the B12 vitamin is a combination of cyanocobalamin and hydroxocobalamin. Vascular diseases that can be treated or prevented using the vitamin compositions of the present invention include, but are not limited to, cardiovascular disease, cerebrovascular disease, peripheral vascular disease, and arteriosclerotic vascular disease. Cardiovascular diseases include, but are not limited to, heart disease, stroke, high blood pressure, coronary artery disease and congestive heart failure. Various forms of dementia associated with vascular disease, such as Alzheimer's disease may also be treated or prevented using the vitamin compositions of the present invention.

Accordingly, it is an object of the present invention to provide a vitamin composition comprising vitamin B12, vitamin B6, folic acid, magnesium and vitamin E.

It is another object of the present invention to provide a vitamin composition comprising vitamin B12, vitamin B6, folic acid, magnesium, vitamin E and niacin.

It is a further object of the present invention to provide a vitamin composition containing vitamin B12 in a hydroxocobalamin form.

It is another object of the present invention to provide a vitamin composition containing vitamin B 12 in a cyanocobalamin form.

It is a further object of the present invention to provide a vitamin composition containing vitamin B12 in a combination of both a hydroxocobalamin form and a cyanocobalamin form.

It is a still further object of the present invention to provide a vitamin composition for the treatment and or prevention of a vascular disease comprising vitamin B12, vitamin B6, folic acid, magnesium and vitamin E.

It is another object of the present invention to provide a vitamin composition for the treatment and or prevention of a vascular disease comprising vitamin B12, vitamin B6, folic acid, magnesium, niacin and vitamin E.

It is a further object of the present invention to provide methods for treating and or preventing a vascular disease or dementia in an individual comprising administering to the individual a vitamin composition comprising vitamin B12, vitamin B6, folic acid, magnesium and vitamin E.

It is a still further object of the present invention to provide methods for treating and or preventing a vascular disease or dementia in an individual comprising administering to the individual a vitamin composition comprising vitamin B12, vitamin B6, folic acid, magnesium, vitamin E and niacin.

It is yet another object of the present invention to provide methods for treating a cardiovascular disease.

It is yet another object of the present invention to provide methods for preventing a cardiovascular disease.

It is yet another object of the present invention to provide methods for treating a cerebrovascular disease.

It is yet another object of the present invention to provide methods for preventing a cerebrovascular disease.

It is yet another object of the present invention to provide methods for preventing or treating dementia, including Alzheimer's disease.

These and other objects, features and advantages of the present invention will become apparent after a review of the following detailed description of the disclosed embodiments and claims.

### DETAILED DESCRIPTION OF THE INVENTION

The present invention provides new and effective vitamin compositions and methods for the treatment and or prevention of vascular diseases and dementia using these vitamin compositions.

Vitamin Compositions

The vitamin compositions of the present invention comprise the following vitamins, vitamin B12, vitamin B6, folic acid, magnesium and vitamin E. In one embodiment, the vitamin composition contains vitamin B12, vitamin B6, folic acid, magnesium, niacin and vitamin E. As used herein, the term “vitamin B12” refers to all forms of cobalamin including, but not limited to, hydroxocobalamin, cyanocobalamin and methylcobalamin. Accordingly, the vitamin compositions of the present invention can include one or more forms of vitamin B12 including hydroxocobalamin, cyanocobalamin and methylcobalamin. In a preferred embodiment, the vitamin compositions include one form of vitamin B12, which form is hydroxocobalamin. In another preferred embodiment, the vitamin compositions include one form of vitamin B12, which form is cyanocobalamin. As also used herein, the term “vitamin B6” includes pyridoxal, pyridoxamine and pyridoxine, and the term “vitamin B3” is synonymous with niacin and nicotinic acid. “Vitamin E” is used herein to refer to alpha-tocopherol, d-alpha-tocopherol, d-alpha-tocopheryl succinate (or acetate), dl-alpha-tocopherol, dl-alpha-tocopheryl acetate (or succinate), gamma tocopherol, mixed tocopherols, mixed tocoretinols, and dl-alpha tocopherol nicotinate. The term “magnesium” is used herein to refer to any form of magnesium including magnesium oxide, magnesium chloride, magnesium lactate, magnesium sulfate and magnesium gluconate. It is also to be understood that as used in the specification and in the claims, “a” or “an” can mean one or more, depending upon the context in which it is used.

The vitamin compositions described herein can contain magnesium in an amount between about 50 mg and 350 mg, preferably between about 75 mg and 150 mg, and more preferably about 100 mg. In another or further embodiment, the vitamin composition contains vitamin E in an amount less than 400 IU, preferably between 50 IU and 350 IU, and more preferably about 100 IU. In another or further embodiment, the vitamin composition contains folic acid in an amount between about 0.8 mg and 5 mg, preferably between about 1 mg and 3 mg, and more preferably about 2 mg, or about 2.05 mg. In another or further embodiment, the vitamin composition contains vitamin B12 in an amount between about 300 mcg and 2000 mcg, preferably between 300 mcg and 1200 mcg and more preferably about 500 mcg. The vitamin B12 may be hydroxy-, cyano- or methylcobalamin, but is preferably hydroxocobalamin. In another or further embodiment, the vitamin composition contains vitamin B6 in an amount between 10 mg and 100 mg and preferably about 25 mg. In another or further embodiment, the vitamin composition contains niacin in an amount less than 250 mg, preferably between about 20 mg and 100 mg, and more preferably about 35 mg.

Accordingly, the vitamin composition may contain one or more of the following amounts of specific ingredients in any combination: magnesium in an amount between about 50 mg and 350 mg, preferably between about 75 mg and 150 mg, and more preferably about 100 mg; vitamin E in an amount less than 400 IU, preferably between 50 IU and 350 IU, and more preferably about 100 IU; folic acid in an amount between about 0.8 mg and 5 mg, preferably between about 1 mg and 3 mg, and more preferably about 2 mg, or about 2.05 mg; vitamin B12 in an amount between about 300 mcg and 2000 mcg, preferably between 300 mcg and 1200 mcg and more preferably about 500 mcg; and vitamin B6 in an amount between 10 mg and 100 mg and preferably about 25 mg. In further embodiments, the vitamin composition may additionally contain niacin in an any amount or an amount less than 250 mg, preferably between about 20 mg and 100 mg, and more preferably about 35 mg.

In one embodiment, vitamin compositions of the present invention comprise one of magnesium, vitamin E, folic acid, vitamin B12 and vitamin B6 in the ranges prescribed above and the other four listed ingredients in any amount. For example, one vitamin composition of the present invention contains magnesium in an amount between about 50 mg and 350 mg, preferably between about 75 mg and 150 mg, and more preferably about 100 mg along with vitamin E, folic acid, vitamin B12 and vitamin B6 in any amount. Another vitamin composition contains vitamin E in an amount less than 400 IU, preferably between 50 IU and 350 IU, and more preferably about 100 IU along with magnesium, folic acid, vitamin B12 and vitamin B6 in any amount. Another vitamin composition contains folic acid in an amount between about 0.8 mg and 5 mg, preferably between about 1 mg and 3 mg, and more preferably about 2 mg, or about 2.05 mg, along with magnesium, vitamin E, vitamin B12 and vitamin B6 in any amount. Another vitamin composition contains vitamin B12 in an amount between about 300 mcg and 2000 mcg, preferably between 300 mcg and 1200 mcg and more preferably about 500 mcg along with magnesium, vitamin E, folic acid and vitamin B6 in any amount. Another vitamin composition contains vitamin B6 in an amount between about 10 mg and 100 mg, and preferably about 25 mg along with magnesium, vitamin E, folic acid and vitamin B12 in any amount. Other vitamin compositions contain magnesium, vitamin E, folic acid, vitamin B12 and vitamin B6 in any amounts or in combinations of amounts described above in addition to niacin in any amount or an amount less than 250 mg, preferably between about 20 mg and 100 mg, and more preferably about 35 mg.

Other vitamin compositions of the present invention comprise two of magnesium, vitamin E, folic acid, vitamin B12 and vitamin B6 in an amount prescribed above and the other three listed ingredients in any amount. Still other vitamin compositions of the present invention comprise three of magnesium, vitamin E, folic acid, vitamin B12 and vitamin B6 in an amount prescribed above and the other two listed ingredients in any amount. Further vitamin compositions comprise four of magnesium, vitamin E, folic acid, vitamin B12 and vitamin B6 in an amount prescribed above and the other one listed ingredient in any amount. In some embodiments, vitamin compositions comprise all five of magnesium, vitamin E, folic acid, vitamin B12 and vitamin B6 in an amount prescribed above. Any of these vitamin compositions may additionally contain niacin in an amount prescribed above.

In one embodiment of the present invention, the vitamin composition contains folic acid in an amount of about 2 mg, hydroxocobalamin in an amount of about 500 mcg, pyridoxine in an amount of about 25 mg, vitamin E (alpha-tocopherol) in an amount of about 100 IU, and magnesium (as oxide) in an amount of about 100 mg. In another embodiment, the vitamin composition contains folic acid in an amount of about 2 mg, hydroxocobalamin in an amount of about 500 mcg, pyridoxine in an amount of about 25 mg, vitamin E (alpha-tocopherol) in an amount of about 100 IU, magnesium (as oxide) in an amount of about 100 mg and niacin in an amount of about 35 mg. In yet another preferred embodiment, the vitamin composition contains folic acid in an amount of about 2 mg, or about 2.05 mg, vitamin B12 (cyanocobalamin in an amount from 1 to 499 mcg and hydroxocobalamin in an amount from 1 to 499 mcg provided that the total amount of cyanocobalamin and hydroxocobalamin does not exceed 500 mcg), vitamin B6 (pyridoxine) in an amount of about 25 mg, vitamin E (alpha-tocopherol) in an amount of about 100 IU, and magnesium (as oxide) in an amount of about 100 mg. In yet another preferred embodiment, the vitamin composition contains folic acid in an amount of about 2 mg, or about 2.05 mg, vitamin B12 (cyanocobalamin in an amount of about 250 mcg and hydroxocobalamin in an amount of about 250 mcg), vitamin B6 (pyridoxine) in an amount of about 25 mg, vitamin E (alpha-tocopherol) in an amount of about 100 IU, and magnesium (as oxide) in an amount of about 100 mg. In some embodiments, the vitamin composition further contains niacin in an amount of about 35 mg.

Auxiliary Agents

The vitamin compositions of the present invention may also contain at least one of any suitable auxiliary such as, but not limited to, diluent, binder, stabilizer, buffers, salts, lipophilic solvents, preservative or the like. Pharmaceutically acceptable auxiliaries are preferred. Examples and methods of preparing such sterile solutions are well known in the art and can be found in well-known texts such as, but not limited to, REMINGTON'S PHARMACEUTICAL SCIENCES (Gennaro, Ed., 18th Edition, Mack Publishing Co. (1990)). Pharmaceutically acceptable carriers can be routinely selected that are suitable for the mode of administration, solubility and/or stability of the compound.

Pharmaceutical excipients and additives useful in the present invention include, but are not limited to, proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination in ranges of 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like.

Carbohydrate excipients suitable for use in the present invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, sorbitol (glucitol), myoinositol and the like.

The vitamin compositions of the present invention can also include a buffer or a pH-adjusting agent. Typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers.

Additionally, vitamin compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, anti-microbial agents, sweeteners, antioxidants, anti-static agents, surfactants (e.g., polysorbates such as “TWEEN 20” and “TWEEN 80”), lipids (e.g., phospholipids, fatty acids), steroids (e.g., cholesterol), and chelating agents (e.g., EDTA). These and additional known pharmaceutical excipients and/or additives suitable for use in the present invention are known in the art, e.g., as listed in REMINGTON: THE SCIENCE & PRACTICE OF PHARMACY (19^{th} ed., Williams & Williams (1995)) and PHYSICIAN'S DESK REFERENCE (52^{nd} ed., Medical Economics (1998)), the disclosures of which are expressly entirely incorporated herein by reference.

Vitamin Formulations for Oral Administration

For oral administration in the form of a tablet or capsule, the vitamins described above (any combination of specified amounts or ranges of vitamin B12, vitamin B6, folic acid, magnesium, vitamin E and optionally niacin) may be combined with an oral, non-toxic pharmaceutically acceptable inert carrier such as ethanol, glycerol, water and the like. Moreover, when desired or necessary, suitable binders, lubricants, disintegrating agents, flavoring and coloring agents may also be incorporated into the mixture. Suitable binders include, without limitation, starch; gelatin; natural sugars such as glucose or beta-lactose; corn sweeteners; natural and synthetic gums such as acacia, tragacanth, or sodium alginate, carboxymethylcellulose; polyethylene glycol; waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

In one embodiment, a vitamin composition suitable for oral administration contains carnauba wax, citric acid, dicalcium phosphate, hydroxypropyl methylcellulose, magnesium stearate, microcrystalline cellulose, polyethylene glycol, polysorbate 80, riboflavin, silicon dioxide, sodium benzoate, sodium citrate, sodium starch glycolate, sorbic acid, starch, stearic acid and titanium dioxide.

Vitamin compositions of the present invention suitable for oral administration may be presented as discrete units such as capsules, cachets or tablets each containing a predetermined amount of a vitamin ingredient; as a powder or granules; as a solution or a suspension in an aqueous liquid or a non-aqueous liquid; or as an oil-in-water liquid emulsion or a water-in-oil emulsion and as a bolus, etc. In a preferred embodiment, the vitamin composition is in the form of a tablet.

A tablet may be made by compression or molding, optionally with one or more accessory ingredients. Such techniques are known to one of ordinary skill in the art. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated so as to provide a slow or controlled release of the vitamin ingredient(s) therein.

Preferred unit dosage formulations are those containing a daily dose or unit, daily sub-dose, or an appropriate fraction thereof, of the administered ingredient. In a more preferred embodiment, the unit dosage formulation contains a daily dose of the vitamin compositions described above.

Vitamin Formulations for Other Routes of Administration

Formulations suitable for parenteral administration include aqueous and non-aqueous sterile injection solutions which may contain anti-oxidants, buffers, bacteriostats and solutes that render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

For parenteral administration, sterile suspensions and solutions are desired. Isotonic preparations, which generally contain suitable preservatives, are employed when intravenous administration is desired. The vitamin compositions may be administered parenterally via injection of a formulation consisting of the active vitamin ingredients dissolved in an inert liquid carrier. The term “parenteral,” as used herein, includes, but is not limited to, subcutaneous injections, intravenous, intramuscular, intraperitoneal injections, or infusion techniques. Acceptable liquid carriers include, for example, vegetable oils such as peanut oil, cottonseed oil, sesame oil and the like, as well as organic solvents such as solketal, glycerol formal and the like. The formulations may be prepared by dissolving or suspending the active ingredient in the liquid carrier such that the final formulation contains from about 0.005% to 30% by weight of the active ingredient, i.e., a vitamin composition of the present invention.

Formulations suitable for topical administration in the mouth include lozenges comprising the ingredients in a flavored basis, usually sucrose and acacia or tragacanth; pastilles comprising the vitamin composition in an inert basis such as gelatin and glycerin, or sucrose and acacia; and mouthwashes comprising the compound to be administered in a suitable liquid carrier. The liquid forms may include suitably flavored suspending or dispersing agents such as the synthetic and natural gums, for example, tragacanth, acacia, methyl-cellulose and the like.

Formulations suitable for nasal administration, wherein the carrier is a solid, include a coarse powder having a particle size, for example, in the range of 20 to 500 microns which is administered in the manner in which snuff is taken, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the vitamin composition.

The compounds may also be entrapped in microcapsules prepared, for example, by coacervation techniques or by interfacial polymerization, for example, hydroxymethylcellulose or gelatin-microcapsules and poly(methylmethacrylate) microcapsules, respectively, in colloidal drug delivery systems (for example, liposomes, albumin microspheres, microemulsions, nano-particles and nanocapsules) or in macroemulsions. REMINGTON'S PHARMACEUTICAL SCIENCES (A. Osol ed., 16th ed. (1980)).

In addition, the vitamin compositions may be incorporated into biodegradable polymers allowing for sustained release of the compound, the polymers being implanted in the vicinity of where vitamin delivery is desired, for example, within a hardened blood vessel such as an artery. The biodegradable polymers and their uses are described, for example, in detail in Brem et al., 74 J. NEUROSURG. 441-46 (1991). Suitable examples of sustained-release compositions include semi permeable matrices of solid hydrophobic polymers containing a compound of the present invention, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT® (Tap Pharmaceuticals, Inc., Chicago, Ill.) (injectable microspheres composed of lactic acid glycolic acid copolymer and leuprolide acetate), and poly-D-(−)-3-hydroxybutyric acid.

Pharmaceutically Acceptable Preservatives

The present invention provides stable vitamin compositions as well as preserved vitamin solutions and formulations containing a preservative as well as multi-use preserved formulations suitable for pharmaceutical or veterinary use, comprising a vitamin composition disclosed herein in a pharmaceutically acceptable formulation. Formulations in accordance with the present invention may optionally contain at least one known preservative. Preservatives include, but are not limited to, phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein. Non-limiting examples include, no preservative, 0.1-2% m-cresol, 0.1-3% benzyl alcohol, 0.001-0.5% thimerosal, 0.001-2.0% phenol, 0.0005-1.0% alkylparaben(s), and the like.

Other excipients, e.g., isotonicity agents, buffers, antioxidants, preservative enhancers, can be optionally added to the diluent. An isotonicity agent such as glycerin, is commonly used at known concentrations. A physiologically tolerated buffer is preferably added to provide improved pH control. The formulations can cover a wide range of pHs, such as from about pH 4.0 to about pH 10.0, specifically, a range from about pH 5.0 to about pH 9.0, and more specifically, a range of about 6.0 to about 8.0. Suitable buffers include phosphate buffers, for example, sodium phosphate and phosphate buffered saline (PBS).

Methods of Treating or Preventing Vascular Disease or Dementia

The present invention further includes methods of treating or preventing a vascular disease or dementia in a human by administering to the individual a vitamin composition comprising vitamin B12, vitamin B6, folic acid, magnesium, vitamin E, and optionally niacin, wherein the amount of the vitamin composition is effective to treat or prevent vascular disease. As used herein, the term “vascular disease” refers to any disease or condition associated with blood vessels such as narrowing of the blood vessels and plaque formation. Vascular disease includes, but is not limited to, cardiovascular disease, cerebrovascular disease, peripheral vascular disease, atherosclerosis, and arteriosclerotic vascular disease. The term “cardiovascular disease” is defined to include, but is not limited to, heart disease, stroke, high blood pressure, coronary artery disease and congestive heart failure.

“Treatment” of a disease does not require a curing of the disease. Accordingly, an “effective amount” is defined herein as an amount capable of reducing the severity or occurrence of one or more conditions associated with a particular disease. For example, in some embodiments, a vitamin composition described herein is administered to a human in an amount effective reduce the occurrence of stroke, reduce the occurrence of congestive heart failure, reduce blood pressure, or reduce the severity of coronary artery disease. A vitamin composition may also be administered to a human in an amount effective to treat one or more of the following conditions: hyperhomocystineamia, low levels of high density lipoprotein (HDL), hypomagnesaemia, and “in-situ” vascular free radical formation. In a preferred embodiment, the vitamin compositions of the present invention treat hyperhomocystineamia, low levels of high density lipoprotein (HDL), hypomagnesaemia, and “in-situ” vascular free radical formation.

Prevention of vascular disease also includes a reduction in the onset or severity of vascular disease in a human by administering the novel vitamin compositions of the present invention. Such prevention may be achieved through administration on a periodic or daily basis, or on any schedule recommended by a health care professional.

Any of the vitamin compositions described herein may be administered to the individual having a vascular disease or dementia. The administered vitamin compositions may contain any combination of the amounts of the following ingredients: magnesium in an amount between about 50 mg and 350 mg, preferably between about 75 mg and 150 mg, and more preferably about 100 mg; vitamin E in an amount less than 400 IU, preferably between 50 IU and 350 IU, and more preferably about 100 IU; folic acid in an amount between about 0.8 mg and 5 mg, preferably between about 1 mg and 3 mg, and more preferably about 2 mg, or about 2.05 mg; vitamin B12 in an amount between about 300 mcg and 2000 mcg, preferably between about 300 mcg and 1200 mcg, and more preferably about 500 mcg; and vitamin B6 in an amount between about 10 mg and 100 mg, and more preferably about 25 mg. The administered vitamin compositions may contain any combination of amounts of magnesium, vitamin E, folic acid, vitamin B12, vitamin B6 and addition to niacin in any amount or an amount less than 250 mg, preferably between about 20 mg and 100 mg, and more preferably about 35 mg.

In one embodiment, the vitamin composition administered to the individual contains vitamin B12, vitamin B6, folic acid, magnesium and vitamin E. In a preferred embodiment, the B12 vitamin is a hydroxocobalamin. In a further preferred embodiment of the present invention, the vitamin composition administered to an individual contains folic acid in an amount of about 2 mg, or about 2.05 mg, hydroxocobalamin in an amount of about 500 mcg, pyridoxine in an amount of about 25 mg, vitamin E (alpha-tocopherol) in an amount of about 100 IU, and magnesium (oxide) in an amount of about 100 mg. In another embodiment, the vitamin composition administered to the individual contains vitamin B12, vitamin B6, folic acid, magnesium, niacin and vitamin E. In a preferred embodiment of the present invention, the vitamin composition administered to an individual contains folic acid in an amount of about 2 mg, or about 2.05 mg, hydroxocobalamin in an amount of about 500 mcg, pyridoxine in an amount of about 25 mg, vitamin E (alpha-tocopherol) in an amount of about 100 IU, magnesium (oxide) in an amount of about 100 mg, and niacin in an amount of about 35 mg.

Routes of Administration

When treating or preventing a vascular disease in an individual, the vitamin compositions disclosed herein may be administered to the individual by any of the following routes: oral, parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, and transdermal means. In a preferred embodiment, a vitamin composition is administered orally.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### EXAMPLE 1

Preparation of Vitamin Composition

A vitamin composition was prepared that contains folic acid USP 2.05 mg, hydroxocobalamin USP 500 mcg, pyridoxine USP 25 mg, vitamin E USP (alpha-tocopherol) 100 IU, and magnesium USP (as oxide) 100 mg. The vitamin composition further contains camauba wax, citric acid, dicalcium phosphate, hydroxypropyl methylcellulose, magnesium stearate, microcrystalline cellulose, polyethylene glycol, polysorbate 80, silicon dioxide, sodium benzoate, sodium citrate, sodium starch glycolate, sorbic acid, starch, stearic acid and titanium dioxide.

All the active vitamin ingredients were sieved through a #20 mesh screen, and lumpy raw materials were removed. Vitamin E was pre-blended with adsorbents, and combined with, folic acid, magnesium oxide, pyridoxine, hydroxocobalamin and fillers in a blender. The materials were blended for 10 to 15 minutes. Additional fillers, disintegrants, glidants and lubricants were added into the blender and the mixture was blended for an additional 10 to 20 minutes. The blend was compressed into tablets according to specifications. The finished tablets were coated in Accella Cota.

### EXAMPLE 2

Administration of Vitamin Composition

The vitamin composition described in Example 1 is indicated for the distinctive nutritional requirements of individuals under physician's treatment with or at risk for: cardiovascular disease, cerebrovascular disease, peripheral vascular disease, arteriosclerotic vascular disease and dementia. Dosage and administration in adults is one tablet daily or as directed by a physician. Accordingly, the vitamin composition described in Example 1 is orally administered to the patients described below in an amount of one tablet daily for various periods. The patients are monitored for reduction of homocysteine levels and or stabilization of plaque determined by echocardiogram, angiogram. Or computerized assisted tomographic (CAT) scan.

Patient 1

Patient is 55 year-old male who entered for screening evaluation. Fasting lipid profile obtained 18 months prior to presentation shows high density lipoprotein (HDL) at 32 mg/dL and total cholesterol (TC) at 251 mg/dL. The patient has a history of unstable angina and is a known hypertensive (systolic blood pressure (SBP) 142). Work-up reveals severe multi-vessel coronary heart disease necessitating 3-vessel coronary artery bypass graft (CABG). Post CABG, the patient completes cardiac rehabilitation, maintains daily aerobic exercise and adopts a strict vegetarian diet (10% calories from fat). The patient is currently on angiotensin converting enzyme (ACE) inhibitor/diuretic and aspirin. Exam otherwise unremarkable.

Administration of the vitamin composition described in Example 1 slightly decreases vascular disease in the graft vessels.

Patient 2

Patient is 70 year-old obese female. The patient has a history of myocardial infarction arrhythmia, which necessitates antithrombolytic therapy followed by angioplasty of the left anterior descending coronary artery and the right coronary artery. Following myocardial infarction and angioplasty, the patient completes cardiac rehabilitation, maintains daily exercise but continues to be obese. The patient is currently receiving a beta-blocker, diuretic, and aspirin.

Administration of the vitamin composition described in Example 1 stabilizes vascular disease in the left anterior descending coronary artery and the right coronary artery and retards the course of vascular disease in the other coronary vessels. Patient has decreased arrhythmic episodes.

Patient 3

Patient is 60 year-old male who suffers from diabetes mellitus II and severe hypertension (systolic pressure 170, diastolic pressure 84). Fasting lipid profile obtained 1 month prior to presentation shows HDL at 34 mg/dL and TC at 421 mg/dL. Patient's familial history is dyslipidemia and heart failure. The patient's diabetes is under control. Patient was previously in a high stress job and recently divorced. Patient is currently on a cholesterol-lowering drug (statin) for three weeks. Patient has also taken Metformin for seven years.

Administration of the vitamin composition described in Example 1 to the patient decreases the progression of vascular disease in the coronary vasculature and in the carotid arteries, thereby decreasing the likelihood of myocardial infarction and stroke. Patient also demonstrates improved diabetes control.

Patient 4

A 47 year-old male who is slightly overweight has a routine annual physical. A family history of myocardial infarction, peripheral vascular claudication, high total serum cholesterol and low density lipoprotein levels are discussed with the physician. The patient has borderline total serum cholesterol levels of 210 mg/dl. A computerized tomographic scan of the heart reveals minimal calcium containing plaque in the coronary vasculature. The physician recommends restriction of dietary fat intake, exercise, a low dose statin, and prescribes a daily oral vitamin described in Example 1. The vitamin therapy helps prevent vascular disease in the coronary vessels, the carotids, the aorta and the popliteal vessels.

Patient 5

A 20 year-old obese male has a routine annual physical. A family history of hyperlipidemia, coronary artery disease, myocardial infarction, and diabetes are discussed with the physician. The patient has total serum cholesterol levels of 300 mg/dl. Electrocadiogram is normal although the patient has limited duration on the treadmill. A computerized tomographic scan of the heart reveals no calcium containing plaque in the coronary vasculature. The physician recommends restriction of dietary fat intake, exercise and prescribes the oral vitamin described in Example 1 at one vitamin per day. The vitamin therapy helps prevent vascular disease in the coronary vessels and retard the onset of myocardial infarction.

Patient 6

Patient is 65 year-old male. The patient has a history of cardiovascular disease and elevated total cholesterol and LDL levels. Scans of the coronary vessels and carotid arteries reveal stenosis and plaque accumulation. The patient is currently receiving a statin, and aspirin.

The patient's physician prescribes daily oral administration of the vitamin composition described in Example 1 to retard the course of vascular disease and to prevent or retard the onset of dementia and Alzheimer's disease.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims.

1. A composition comprising vitamin B12, vitamin B6, folic acid, magnesium, and vitamin E.

2. The composition of claim 1, wherein the magnesium is in an amount between about 50 mg and 350 mg.

3. The composition of claim 1, wherein the vitamin E is an amount less than 400 IU.

4. The composition of claim 1, wherein the vitamin E is in an amount between about 50 IU and 350 IU.

5. The composition of claim 1, wherein the folic acid is in an amount between about 0.8 mg and 5 mg.

6. The composition of claim 1, wherein the vitamin B12 is hydroxocobalamin.

7. The composition of claim 6, wherein the hydroxocobalamin is in an amount between about 300 mcg and 2000 mcg.

8. The composition of claim 1, wherein the vitamin B12 is cyanocobalamin.

9. The composition of claim 8, wherein the cyanocobalamin is in an amount between about 300 mcg and 2000 mcg.

10. The composition of claim 1, wherein the vitamin B12 is a mixture of cyanocobalamin and hydroxocobalamin.

11. The composition of claim 10, wherein the cyanocobalamin is in an amount of between about 300 mcg and 2000 mcg and the hydroxocobalamin is in an amount of between about 300 mcg and 2000 mcg, provided the amount of cyanocobalamin and the amount of hydroxocobalamin does not exceed 2000 mcg.

12. The composition of claim 1, wherein the vitamin B6 is in an amount of between about 10 mg and 100 mg.

13. The composition of claim 1, wherein magnesium is in an amount between about 50 mg and 350 mg, vitamin E is in an amount between about 50 IU and 400 IU, folic acid is in an amount between about 0.8 mg and 5 mg, vitamin B12 is in an amount between about 300 mcg and 2000 mcg, and vitamin B6 is in an amount between about 10 mg and 100 mg.

14. The composition of claim 1, wherein magnesium is in an amount of about 100 mg, vitamin E is in an amount of about 100 IU, folic acid is in an amount of about 2.05 mg, vitamin B12 is in an amount of about 500 mcg, and vitamin B6 is in an amount of about 25 mg.

15. The composition of claim 1 further comprising niacin.

16. The composition of claim 15, wherein the niacin is in an amount less than 250 mg.

17. The composition of claim 15, wherein the niacin is an amount of about 35 mg.

18. A method of treating or preventing a vascular disease or dementia in a human comprising administering to the human an amount of a composition comprising vitamin B12, vitamin B6, folic acid, magnesium and vitamin E, wherein the amount is effective to treat or prevent the vascular disease or the dementia in the human.

19. The method of claim 18, wherein the vitamin B12 is hydroxocobalamin.

20. The method of claim 18, wherein the vitamin B12 is cyanocobalamin.

21. The method of claim 18, wherein magnesium is in an amount between about 50 mg and 350 mg, vitamin E is in an amount between about 50 IU and 400 IU, folic acid is in an amount between about 0.8 mg and 5 mg, vitamin B12 is in an amount between about 300 mcg and 2000 mcg, and vitamin B6 is in an amount between about 10 mg and 100 mg.

22. The method of claim 18, wherein magnesium is in an amount of about 100 mg, vitamin E is in an amount of about 100 IU, folic acid is in an amount of about 2.05 mg, vitamin B12 is in an amount of about 500 mcg, and vitamin B6 is in an amount of about 25 mg.

23. The method of claim 18, wherein the composition further comprises niacin.

24. The method of claim 18, wherein the vascular disease is a cardiovascular disease, cerebrovascular disease, peripheral vascular disease, an atherosclerotic vascular disease or an arteriosclerotic vascular disease.

25. The method of claim 18, wherein the vascular disease is a cardiovascular disease.

26. A composition comprising hydroxocobalamin, vitamin B6, folic acid, magnesium, and vitamin E.

27. A method of treating or preventing a vascular disease or dementia in a human comprising administering to the human an amount of a composition comprising hydroxocobalamin, vitamin B6, folic acid, magnesium and vitamin E, wherein the amount is effective to treat or prevent the vascular disease or the dementia in the human.
